(19) United States
(12) Patent Application Publication
(10) Pub. No.: US 2026/0337111 A1
(43) Pub. Date: Oct. 29, 2026

(54) Advanced Ceramic Emitting Nonthermal Infrared Radiation in the 3-16 µm Range for Broad Applications

(71) Applicant: Albert Chin-Tang WEY, Westmont, IL (US)

(72) Inventor: Albert Chin-Tang WEY, Westmont, IL (US)

(21) Appl. No.: 19/727,654

(22) Filed: Jul. 1, 2026

Related U.S. Application Data
(63) Continuation-in-part of application No. 18/199,660, filed on May 19, 2023.
(63) Continuation-in-part of application No. 19/191,779, filed on Apr. 28, 2025.

(51) Int. Cl. C04B 35/14; C04B 35/64; C08K 3/36; D06M 11/38; D06M 11/44; D06M 11/45; D06M 11/49; D06M 11/79

(52) U.S. Cl. CPC C04B 35/14; C04B 35/64; C08K 3/36; D06M 11/38; D06M 11/44; D06M 11/45; D06M 11/49; D06M 11/79; C04B 2235/3201; C04B 2235/3208; C04B 2235/3217; C04B 2235/3241; C04B 2235/3274; C04B 2235/3418; C04B 2235/656; D06M 2200/00

(57) ABSTRACT

The present invention relates to a ceramic composite configured to emit nonthermal infrared photons in the 3-16 μm wavelength range through a crystal structure that enables electron transitions under crystal field splitting, distinguishing it from conventional heat-based far-infrared radiation in the 3-1000 m range. The ceramic composite is suitable for a variety of applications, including therapeutic devices for humans and animals, in which the emitted 3-16 m wavelength photons selectively induce molecular vibrations that enhance chemical reactions. By promoting intracellular vibrational activity, these nonthermal infrared photons may support natural healing processes and help improve physiological function, immune response, overall health, and longevity.

### CROSS REFERENCE TO RELATED APPLICATIONS

This is a Continuation-in-Part (CIP) claiming the benefit of U.S. patent application Ser. No. 18/199,660, filed on May 19, 2023, and Ser. No. 19/191,779, filed on Apr. 28, 2025. These applications are incorporated by reference herein in their entireties.

### BACKGROUND OF THE INVENTION

### 1. Field of the Invention

The invention relates to a ceramic material that emits nonthermal infrared (IR) photons in the 3-16 μm wavelength range for a wide range of applications, including education, agriculture, automotive products, industrial uses, clothing, water and beverage treatment, household goods, and therapy. As used herein, “nonthermal IR” means infrared radiation produced by non-heat mechanisms, particularly electron transitions described by Crystal Field Theory. By contrast, prior-art thermal far-infrared (FIR) radiation is driven by temperature, spans about 3-1000 μm, and mainly transfers heat. Unlike thermal FIR, the nonthermal IR photons of the present invention do not heat objects they contact. Instead, they selectively excite molecular vibrations to produce specific chemical and biological effects, enabling targeted functions not achievable with conventional heat-based FIR materials.

### 2. Background Art

As used herein, the term “nonthermal infrared photon emission” (or simply “nonthermal IR”) refers to the emission of photons from a crystal structure comprising transition metal ions incorporated within coordination complexes and subject to crystal field splitting in accordance with Crystal Field Theory (CFT).

In such crystal structures, permitted vibronic transitions occur across an energy gap of approximately 100-315 meV, corresponding to infrared photon emission at wavelengths of about 4-12 μm and producing a luminescence profile spanning approximately 3-16 μm. The relationship between energy and wavelength is expressed by the formula:

E (eV)=1.2398/λ(μm)
where E represents the energy and
λ represents the wavelength, generally measured in micrometers or microns in μm.

Such emission is fundamentally distinct from prior-art FIR thermal radiation with respect to origin, mechanism, and resulting effect.

At room temperature, all objects emit thermal electromagnetic radiation, with the predominant spectral power distribution falling within the infrared region and commonly extending over wavelengths of approximately 3 to 1000 μm. Such thermal emission is governed by Planck's law, which defines the spectral radiance of blackbody radiation as a function solely of absolute temperature. A blackbody is an idealized body that completely absorbs incident electromagnetic radiation and, at thermal equilibrium, emits radiation with a spectral distribution determined exclusively by its temperature, consistent with Kirchhoff's law of thermal radiation.

Technically, the 3-16 μm region falls within the mid-infrared spectrum, while the far-infrared range covers wavelengths from about 15 μm to 1,000 μm. However, industrial convention typically defines FIR as encompassing the entire 3-1000 μm range of thermal radiation. This broader categorization likely stems from practical considerations in industrial applications, where the distinction between mid- and far-infrared is less relevant. Despite this technical distinction, the 3-16 μm range is still commonly referred to as FIR in the field due to this industrial convention.

In view of that, a frequent error in prior art occurs when assuming that thermal radiation can be selectively emitted only as “3-16 m” wavelength photons at room temperature. According to Planck's Law, an object at room temperature emits radiation across the entire 3-1000 μm FIR range, with approximately 67% of this emission occurring between 3-16 am. Nonetheless, blackbody radiation (thermal radiation) has an inherently broad wavelength range and is not confined to this narrow segment. This misunderstanding can lead to incorrect design assumptions in FIR applications, potentially limiting their effectiveness in targeted applications.

Prior art often characterizes FIR-emitting materials using thermal radiation-related aspects like “heat energy” and “emissivity,” since emissivity indicates the effectiveness of a material in emitting energy as thermal radiation. The emissivity of a surface depends on its chemical composition and geometrical structure. Quantitatively, it is the ratio of the thermal radiation from a surface to the radiation from an ideal black surface at the same temperature as given by the Stefan-Boltzmann Law. For instance, some prior art patents and applications (e.g., U.S. Pat. Nos. 8,285,391, 9,308,388, 9,962,441, and Published Patent Application Nos. 2023/0271028, 2025/0179357, among others) describes FIR materials primarily in terms of their surface emissivity and heat energy transfer, thereby reflecting the erroneous concept that the selective emission properties of FIR materials can be fully described by thermal characteristics, rather than recognizing the distinct mechanisms involved in nonthermal IR emission within the 3-16 μm range.

U.S. Pat. No. 8,285,391 discloses hygienic, therapeutic, and personal care devices that use advanced far-infrared (FIR) radiation technology. The devices incorporate specially engineered conductive materials that generate far-infrared radiation at intensities ranging from 10 to thousands of times greater than conventional conductive materials. These materials also convert energy to radiation with very high efficiency, typically exceeding 90%. The radiation properties of the proposed conductive materials do not follow the Wien's law (“black body” law).

U.S. Pat. No. 9,308,388 discloses ceramic footbath boots and the specialized ceramic materials used to manufacture them. The device consists of boot-shaped footbath containers whose inner surfaces contain textured net-like patterns and raised papillary structures positioned to correspond with foot reflexology zones. The ceramic materials, preferably tourmaline-containing silicates, are designed to provide several therapeutic effects, including: Emitting far-infrared radiation in the 4-14 micron wavelength range with an emissivity greater than 0.92 at room temperature, continuously releasing negative ions, generating weak static electrical currents intended to mimic the body's natural bioelectric currents, and releasing minerals and trace elements into the bath water.

U.S. Pat. No. 9,962,441 primarily discloses a method for improving sleep quality by placing against a person's skin an article coated with a bioceramic composition that emits far-infrared (FIR) thermal radiation when exposed to body heat or another heat source. The bioceramic coating consists essentially of: 40-60 wt % kaolinite (greater than 55% purity), 5-15 wt % tourmaline (greater than 55% purity), 15-25 wt % aluminum oxide (Al₂O₃), and 10-20 wt % silicon dioxide (SiO₂). The claimed benefit is that the far-infrared radiation generated by the coating enhances the quality of sleep for the user. The invention is directed both to the specific bioceramic composition and its use in sleep-improvement products that contact the skin.

US Published Patent Application No. 2023/0271028 primarily discloses a portable far-infrared (FIR) radiation device that uses an electrically conductive, heat-generating layer sandwiched between two flexible polymeric sheet layers made from materials such as PVC, rubber, neoprene, polyurethane, or synthetic resins. The conductive layer contains conductive carbon powder, germanium, polyurethane resin, a coagulating agent, and a diluent, and includes an electrical power inlet. When powered, the conductive carbon powder converts electrical energy into heat, which generates FIR radiation. The conductive layer is formulated with approximately 14-18 wt % conductive carbon powder, 0.5-2.5 wt % germanium, 3-6 wt % coagulating agent, 15-35 wt % polyurethane resin, and 45-66 wt % diluent, creating a flexible FIR-emitting structure suitable for portable therapeutic, wellness, and personal care applications.

Published Patent Application No. 2025/0179357 primarily discloses a far-infrared (FIR) emitting material and a method for its preparation. The material is formed from zirconium monoxide combined with one or more natural silicate mineral soils. The raw materials are mixed in selected proportions, sintered at temperatures between 1100 °C and 1250 °C, and processed into granules or powder. Preparation involves ball milling the materials into a slurry, optionally adding dispersing additives, followed by sifting, baking, drying, and final screening. The resulting FIR-emitting material exhibits high emissivity and emits radiation across a broad wavelength range of approximately 8-20 μm, making it suitable for applications requiring efficient far-infrared radiation.

In contrast to thermal radiation (as represented by various disclosures discussed above), which arises from temperature-dependent energy emission, nonthermal IR emission results from specific electron transitions and is not directly governed by heat at the surface. Although thermal radiation, extending over approximately 3-1000 μm, and nonthermal emission, occurring within approximately 3-16 μm, may overlap in wavelength, the respective mechanisms responsible for such emissions are fundamentally distinct. This distinction highlights the limitations of relying solely on thermal parameters to describe the behavior and effectiveness of FIR materials of prior art in applications where nonthermal IR emission is critical.

For example, chemistry textbooks explain that infrared photons within the 3-16 m wavelength range can be absorbed by molecules, causing them to undergo specific vibrational modes, such as stretching or bending. When molecules absorb these photons, their vibrational energy levels increase, raising the internal energy of the reactants. This elevated internal energy can make it easier for reactants to overcome the Activation Energy. As a result, the Activation Barrier is reduced, leading to enhanced reaction rates.

In practice, biochemical reaction rates may increase by different amounts depending on whether the system is exposed to thermal radiation or to specific IR photons in the 3-16 μm range. These reaction rates are described by the Arrhenius Equation, which relates reaction rate (k) to temperature (T) and activation energy (E_{A}):

k=A exp (−E_{A}/RT)
where k is the rate constant (frequency of collisions resulting in a reaction),
T is the absolute temperature in Kelvins,
A is the pre-exponential factor or Arrhenius factor or frequency factor,
E_{A} is the molar activation energy for the reaction, and
R is the universal gas constant.

Absorbing thermal radiation can raise body temperature. As temperature (T) rises, the reaction rate (k) also increases. Near room temperature, many reactions approximately double in rate for every 10 °C (or 10° K) increase. Therefore, in therapeutic applications where body or skin temperature rises by about 1-5 °C, reaction rates may increase by roughly 10-50%.

In contrast, the absorption of IR photons in the 3-16 μm wavelength range by molecules leads to vibrational excitation, which lowers the activation energy (E_{A}) required for chemical reactions, by approximately 8-40 KJ. The relationship is described by the formula E_{λ} (KJ)=120 KJ/λ(μm), which quantifies the photon energy based on wavelength. Under these conditions, reaction rates can increase exponentially, sometimes by as much as 50 to 200 times.

This remarkable effect is in fact the motivating force behind various inventions of the present inventor, as described in US Pat. Pub. Nos. 2023/0080964, 2023/0322627, and unpublished U.S. patent application Ser. No. 19/191,779, which aim to develop advanced ceramic materials capable of emitting nonthermal IR photon radiation, specifically in the 3-16 m wavelength spectrum known for its nonthermal chemical and biological effects. The disclosures of these US patent applications are incorporated by reference herein, where appropriate

Among these inventions, US Pat. Pub. No. 2023/0080964 was the first to identify an inhomogeneous (polar) crystalline structure. In this structure, transition metal (TM) ions are positioned within a localized electric field, forming specialized sites for the emission of photons in the 3-16 μm range.

This discovery was followed by a continuation-in-part (CIP) invention, published as US Pat. Pub. No. 2023/0322627. In this invention a solid solution is used, in which silicon oxide and aluminum oxide serve as the solvent, while iron oxide and/or chromium oxide act as the solute. The resulting ceramic composite forms a crystal structure that includes an octahedral complex or a tetrahedral complex, which serve as the luminescence centers for emitting 3-16 μm IR photons. Another CIP invention, unpublished U.S. patent application Ser. No. 19/191,779, filed on Apr. 28, 2025 by a common inventor, further identifies that a polymerized silicate structure, preferably a phyllosilicate containing distorted octahedral coordination complexes, is required for emitting nonthermal IR photons, in addition to low dosage ionizing radiation. This structure can be formed by a mixture including at least 8 wt. % silicon oxide, at least 2 wt. % aluminum oxide, and at least 2 wt. % chromium oxide or iron oxide, along with charge-balancing cations and radioactive elements.

### SUMMARY OF THE INVENTION

Based on the foregoing research, the present invention discloses a composition configured to emit energetic nonthermal IR photons in the absence of radioactive elements. The present invention discloses a minimum composition sufficient to form a ceramic composite capable of emitting such nonthermal IR photons.

This composition requires three key components: at least 20 wt. % silicon oxide; at least 8 wt. % aluminum oxide together with at least 2 wt. % iron oxide and/or chromium oxide; and at least 2 wt. % metal oxide. Following calcination, the composite forms at least a crystal structure comprising a tetrahedral layer of silicon oxide and an octahedral layer made up of aluminum oxide together with iron oxide and/or chromium oxide. These layers are interconnected through shared oxygen atoms, forming coordination complexes. The metal elements from metal oxides, along with the oxygen atoms, provide a localized electrical field that causes crystal field splitting, ensuring the emission of nonthermal IR radiation within the 3-16 μm wavelength spectrum.

Since nonthermal IR emission is both original and unprecedented, the present inventor has also developed a novel measurement method as described in separate inventions, including those disclosed in US Pat. Pub. Nos. 2024/0003747, 2025/0314531. This method verifies the emission of nonthermal IR radiation specifically within the 3-16 μm range. Typically, nonthermal IR radiation is hidden by the much stronger thermal radiation present in conventional settings and therefore is undetectable. This advancement is significant because it enables the identification of faint nonthermal IR signals that would otherwise go unnoticed. As a result, this technique could provide evidence in situations where distinguishing nonthermal IR emissions from known thermal radiation is essential.

In summary, much of the FIR-related prior art relies on broad thermal radiation across a wavelength range of 3-1000 μm, yet often claims selective range of 3-16 μm emission, without material properties capable of such targeted output. By contrast, the present invention uses electron transitions described by Crystal Field Theory to generate nonthermal IR emission at specific wavelengths within the 3-16 μm spectrum, thereby distinguishing the invention from conventional thermal-emission (i.e., FIR) methods of the prior art.

Accordingly, one feature of this invention is to provide a ceramic composite that emits nonthermal IR photons in the 3-16 μm wavelength range through electron transitions rather than heat.

Another feature of the present invention is to provide a ceramic composite for applications, including therapeutic uses that benefit from molecular excitation and enhanced chemical reaction rates.

Another feature of the present invention is to provide a ceramic composite capable of emitting nonthermal IR photons in the 3-16 μm wavelength range for incorporation into a therapeutic device to improve health conditions in humans or animals.

Another feature of the present invention is to provide a simple, effective, user-friendly, maintenance-free therapeutic device that can be flexibly attached to any part of the human or animal body requiring treatment.

These features are achieved by a ceramic composite that emits nonthermal infrared photons in the 3-16 μm range. The composite is made by calcining selected oxide or mineral powders to form an engineered crystal structure based on Crystal Field Theory. It can be produced in various forms for broad applications, including therapeutic use.

Other features, objects and advantages of the present invention will hereinafter become apparent to those skilled in the art from the following description.

According to the present invention, a ceramic composite is formed by calcining a mixture comprising selected oxide and/or mineral powders and bonding agents in accordance with a predetermined process. The ceramic composite thus formed comprises a crystal structure exhibiting crystal field splitting sufficient to facilitate electron transitions that emit nonthermal infrared photons having wavelengths in the range of about 3-16 μm. In contrast to thermal far-infrared radiation, the nonthermal infrared photons emitted by the ceramic composite do not substantially heat objects placed in contact therewith, but instead selectively excite molecular vibrations so as to induce desired chemical and biological effects. Accordingly, the ceramic composite is capable of providing targeted functionalities not attainable by conventional heat-based far-infrared materials and is thereby distinguishable from prior-art thermal emission systems.

### BRIEF DESCRIPTION OF THE DRAWINGS

FIG. 1 is a schematic view illustrating a crystal structure according to the present invention, the crystal structure comprising alternating silica tetrahedral sheets and alumina octahedral sheets, wherein cations are uniformly distributed within an interstitial layer disposed between the sheets.

FIG. 2 is a top perspective view of a first embodiment of the present invention, illustrating a plurality of ceramic composites mounted on a flexible substrate configured for attachment to a body part to be treated, wherein each ceramic composite includes a concave surface oriented toward the body.

FIG. 3 is a view of a second embodiment of the present invention, illustrating a ceramic composite, or ceramic particles, formed in a spherical configuration.

FIG. 4 is a view of a third embodiment of the present invention, illustrating a ceramic composite, or ceramic particles, formed in a circular plate configuration.

FIG. 5 is a view of a fourth embodiment of the present invention, illustrating a ceramic composite, or ceramic particles, formed in a rectangular plate configuration.

FIG. 6 is a view of a fifth embodiment of the present invention, illustrating a ceramic composite, or ceramic particles, formed in a rod or wire configuration.

FIG. 7 is a view of a sixth embodiment of the present invention, illustrating a rectangular base material coated with ceramic particles of the present invention.

FIG. 8 is a view of a seventh embodiment of the present invention, illustrating a wire or fiber coated with ceramic particles of the present invention.

Reference Numerals in Drawings

| | |
|---|---|
| 11 ceramic composite | 12 substrate |
| 13 base material | 14 fiber or wire |
| 21 oxygen atom | 22 silicon atom |
| 23 aluminum atom | 24 charge-balancing cation |
| 31 ceramic particles | |

### DETAILED DESCRIPTION OF THE PREFERRED EMBODIMENTS

The ceramic material of the present invention is designed to emit nonthermal IR photons in the 3-16 μm range through a crystal structure that creates crystal field splitting and enables the necessary electron transitions within the d-orbitals of transition metal ions in coordination complexes. In contrast, conventional FIR ceramics merely rely on thermal radiation, which typically spans wavelengths of about 3-1000 m.

All objects above absolute zero emit thermal radiation. This occurs because thermal motion causes charged particles (such as electrons, ions, and molecular dipoles) to accelerate, and accelerating charges emit electromagnetic energy. The combined effect of these motions produces the familiar blackbody spectrum described by Planck's law.

Most prior-art FIR inventions rely on thermal radiation and attempt to increase output in the “3-16 m” range by modifying material emissivity, a measure of how effectively a material emits thermal radiation. As defined above Emissivity (F) is the ratio of radiation emitted by a real surface to that emitted by an ideal blackbody at the same temperature, ranging from 0 for a perfect reflector to 1 for a perfect emitter.

A material's emissivity is determined by a limited set of physical factors, including composition, surface condition, atomic structure, electron density, and bonding characteristics. Accordingly, different materials emit infrared radiation with different efficiencies. Most prior art attempted to increase thermal-radiation emissivity by varying oxide compositions and calcination temperatures to approach the ideal value of 1.0, but typically achieved only about 0.85-0.95.

In therapeutic applications, the Arrhenius equation indicates that biochemical reaction rates can increase in two ways: by raising reactant temperature through heat therapy (thermal radiation) or by lowering the activation barrier through absorption of infrared photons in the 3-16 μm range. Unlike prior art, which depends on thermal radiation and offers limited effects, the present invention focuses on ceramic materials that emit nonthermal IR photons in the wavelength range of 3-16 μm because of their substantial therapeutic potential.

Through years of research, the present inventor first identified in US Pat. Pub. No. 2023/0080964 an inhomogeneous (polar) crystalline structure in which transition metal ions, situated within a localized electric field, act as luminescence centers that emit photons in the 3-16 μm wavelength range. The structure is formed from selected oxide powders that serve as the host, photon-emitting, and cation-doping components of the FIR composite system. By breaking crystal symmetry and creating a polar, inhomogeneous environment, the system permits atomic radiation from orbitals 4s-3d (or 5s-4d) and related hybrid-orbital transitions in the transition metal ions that would otherwise be forbidden by the known Selection Rules.

This discovery formed the basis of a continuation-in-part (CIP) invention, published as US Pat. Pub. No. 2023/0322627, which discloses a solid solution in which silicon oxide and aluminum oxide act as the solvent and iron oxide and/or chromium oxide act as the solute. The resulting ceramic composite forms a crystal structure containing octahedral and/or tetrahedral complexes that serve as luminescence centers for emitting 3-16 μm IR photons.

Another CIP invention (aforementioned unpublished commonly invented U.S. patent application Ser. No. 19/191,779) further shows that emitting nonthermal IR photons together with low-dose ionizing radiation requires a polymerized silicate structure, preferably a phyllosilicate with distorted octahedral coordination. This structure includes at least 8 wt. % silicon oxide, at least 2 wt. % aluminum oxide, and at least 2 wt. % chromium oxide or iron oxide, along with charge-balancing cations and radioactive elements.

Based on this research, the present inventor has developed a composition that emits strong nonthermal IR photons without radioactive elements. The invention also discloses a minimum composition for forming a ceramic composite that emits nonthermal IR photons in the 3-16 μm wavelength range. For such emissions to occur, the polycrystalline material must contain crystalline regions large enough to support the nonthermal IR emission mechanism, which requires transition metal ions such as iron (Fe³⁺) and/or chromium (Cr³⁺) in distorted octahedral complexes.

The ceramic composite of the present invention is made from three powder sets. The first contains at least 20 wt. % silicon oxide (SiO₂). The second contains at least 8 wt. % aluminum oxide (Al₂O₃) together with at least 2 wt. % iron oxide (Fe₂O₃), chromium oxide (Cr₂O₃), or both. The third contains at least 2 wt. % metal oxide, which may include an oxise from one or more metallic elements such as lithium (Li), sodium (Na), potassium (K), magnesium (Mg), calcium (Ca), titanium (Ti), manganese (Mn), cobalt (Co), nickel (Ni), copper (Cu), zinc (Zn), zirconium (Zr), and niobium (Nb) to enhance crystal field properties.

More specifically, the composite is formulated and processed to form a polycrystalline structure that includes at least one polymerized silicate with distorted octahedral compounds. The polymerized silicate may be a chain silicate, sheet silicate, or framework silicate, although a phyllosilicate structure is preferred.

The phyllosilicate structure comprises alternating silica tetrahedral and alumina octahedral sheets. In the silica sheet, each silicon atom is bonded to four oxygen atoms (Si—O₄), while in the alumina sheet, each aluminum atom is coordinated with six oxygen atoms in an octahedral arrangement (Al—O₆). The two sheets are linked by shared oxygen atoms, and charge-balancing cations (metal ions) are uniformly distributed within the interstitial layer.

During calcination, Cr³⁺ and Fe³⁺ replace Al³⁺ at octahedral sites through a charge-coupled substitution mechanism, thereby forming distorted octahedral compounds. Because the formation energies are negative for both Fe (−0.58 eV) and Cr (−0.27 eV), these atoms readily substitute for Al in the phyllosilicate structure. Upon substitution, Fe and Cr exist as trivalent cations, Fe³⁺ and Cr³⁺, within the structure.

The phase transition starts at about 950 °C, but calcination above 1100 °C is required to form the essential distorted octahedral compound. As temperature and dopant concentration (Cr³⁺ and Fe³⁺) increase, the lattice constant decreases because of densification and structural rearrangement. This contraction is attributed to the different ionic radii of Cr³⁺ (0.615 Å) and Fe³⁺ (0.550 Å) relative to Al³⁺ (0.535 Å) at octahedral sites. The process is also highly sensitive to local disorder and substitutional inhomogeneity, which affect the structural evolution of the materials.

The composition is further processed to create a strong-field, low-spin environment within the phyllosilicate structure, consistent with Crystal Field Theory (CFT). In this environment, each transition-metal coordination complex, [CrO₆]³⁺ or [FeO₆]³⁺, serves as a persistent luminescence center for 3-16 μm photons through electron transitions between d-orbital energy levels that would otherwise be forbidden by the Selection Rule. As a result, the ceramic composite emits nonthermal photons in the 3-16 μm wavelength range.

Under Molecular Orbital (MO) theory, transition metal-ligand (M-L) bonding in octahedral transition-metal complexes [ML₆] is understood by examining molecular orbitals formed by overlap between metal 3d orbitals and ligand orbitals. Crystal Field Theory complements this analysis by describing splitting of the d orbitals and the resulting electronic structure of transition-metal (TM) complexes, including their optical and magnetic properties.

A free TM ion has five degenerate d orbitals. In an octahedral TM complex, six ligands are arranged along the x, y, and z axes, causing the dₓ_{²}_{y}_{²} and d_{z}_{²} orbitals that point directly toward the ligands to interact most strongly and rise in energy. These two orbitals together form the e_{g} set, named for the symmetry of the octahedral field.

By contrast, the other three d orbitals (d_{xy}, d_{xz}, and d_{yz}) lie between the ligand bond axes rather than pointing directly toward them. Consequently, they interact less strongly with the donor electrons and collectively form the t_{2g} set.

In an octahedral complex, six negatively charged ligands surrounding a TM ion split the five degenerate d orbitals into two energy levels: the higher-energy e_{g} set and the lower-energy t_{2g} set. This effect is known as crystal field splitting. Although the total energy of the d orbitals is conserved, the redistribution of energy among these orbitals can change the electronic energy depending on how the electrons occupy the e_{g} and t_{2g} levels.

The degeneracy of the e_{g} and t_{2g} orbitals may be further lifted by coupling to the lattice, an effect known as the Jahn-Teller (J-T) distortion effect. In a “Z-in” J-T distortion, shortening the bonds along the z-axis raises the energies of the orbitals with z components, d_{z}_{²}, d_{xz}, and d_{yz}, relative to dₓ_{²}_{-y}_{²} and dy.

One planned FIR luminescence center in the present invention is [FeO₆]³⁺, an iron(III) ion (Fe³⁺) coordinated by six oxygen anions. In forming this complex, Fe³⁺ uses six hybrid orbitals derived from the 4s, 4p, and 4d levels (sp³d²) to accept lone-pair electrons from the oxygen ligands.

The FIR luminescence mechanism is based on Fe³⁺, which has a 3d⁵ electronic configuration at an octahedral site. Calcination produces a crystal geometry that induces a “Z-in” Jahn-Teller distortion, lifting the degeneracy within the t_{2g} set and lowering the energy of the d_{xy} orbital relative to the d_{xz} and d_{yz} orbitals by an energy difference δ₂. The designed δ₂ is about 100 meV, and the associated vibronic transitions are about 100-315 meV, corresponding to λ 4-12 μm and a photoluminescence profile spanning the 3-16 μm band.

Vibrational energies are determined by the following relation:

$${E}_{v} = ( v + \frac{1}{2} ) ℏω - {( v + \frac{1}{2} )}^{2} \frac{ℏω {x}_{e}}{2} + …$$

The Each electronic state of a molecule has a ladder of quantized vibrational levels, with its own equilibrium geometry, vibrational frequency co, and anharmonicity xₑ. As a result, excited states generally have different vibrational level spacings than the ground state. The vibrational energies of excited states are obtained by analyzing vibronic transitions within an electronic absorption or emission band and assigning the peak spacings to the vibrational level spacings of the excited state, as guided by Franck-Condon overlaps between ground- and excited-state vibrational wavefunctions.

After calcination, under the strong-field, low-spin condition for [FeO₆]³⁺, two of the five electrons occupy the lower-energy d_{xy} orbital, while the other three occupy the higher-energy d_{xz} and d_{yz} orbitals.

When the ceramic composite absorbs ambient thermal radiation, that energy is converted into phonons, or lattice vibrations. These vibrations generate heat, raise the composite's temperature, and cause it to emit thermal radiation until it reaches thermodynamic equilibrium with its surroundings, as in prior-art FIR ceramics. At the same time, they induce dynamic Jahn-Teller distortions in the transition-metal octahedra, driving repeated shifts between the “Z-in” and “Z-out” configurations.

Under the Laporte Selection Rule, d-d transitions are forbidden in centrosymmetric complexes such as ideal octahedral complexes. However, when the central TM ion is in a distorted field, molecular vibrations can temporarily break centrosymmetry. These odd ligand vibrations create a transient distorted field that persists long enough (about 10⁻¹⁵ s) for electronic transitions to occur, consistent with the Franck-Condon principle.

As noted above, in the “Z-in” configuration, two electrons occupy the lower-energy d_{xy} orbital and three occupy the higher-energy d_{xz} and d_{yz} orbitals. When the structure switches to the “Z-out” configuration, these energy levels invert, making d_{xz} and d_{yz} lower in energy than d_{xy}. One electron then transitions from d_{xy} to the lower-energy d_{xz} or d_{yz} orbital, emitting a 3-16 μm photon with energy corresponding to δ₂.

Because lattice vibrations occur on the order of 1013 s while electronic transitions occur on the order of 10⁻¹⁶ s, the Franck-Condon principle is satisfied: the electronic transition is effectively vertical, with negligible change in nuclear configuration.

Over time, the system returns from the “Z-out” to the “Z-in” configuration. During this transition, one electron moves from the higher-energy d_{xz} or d_{yz} orbital to the lower-energy d_{xy} orbital, emitting a photon in the 3-16 μm range. This cycle repeats as the system alternates between the “Z-in” and “Z-out” states, causing the ceramic composite to emit IR photons spontaneously and continuously as long as the emission conditions are maintained.

An important requirement is that the ceramic composite be processed above 1100 °C to establish the strong-field, low-spin environment needed to form the intended 3-16 μm infrared luminescence centers, [FeO₆]³⁺.

Fe³⁺ has a 3d⁵ electronic configuration, with five d electrons singly occupying the degenerate 3d orbitals. Their spins align in parallel according to Hund's rule, which favors the lowest-energy state with the maximum total spin. The pairing energy P is the energy required to place two electrons in the same orbital because of electron-electron repulsion. For 3d transition metals, P is typically about 15,000 cm⁻¹ (1.86 eV). Thus, a complex is “strong-field and low-spin” when Δₒ>P, and “weak-field and high-spin” when Δₒ<P.

Shortening of the Fe—O bonds in [FeO₆]³⁺ increases electron repulsion and drives the Fe³⁺ octahedral site from a high-spin (HS) to a low-spin (LS) state. In this LS configuration, the crystal-field splitting energy is about 22,200 cm⁻¹, corresponding to Δₒ≈2.70 eV (21,800 cm⁻¹) between the centers of the t_{2g} and e_{g} bands, which is characteristic of octahedrally coordinated LS Fe³⁺. This LS Fe³⁺ state is essential to the persistent luminescence mechanism that enables 3-16 μm photon emission in the ceramic composite of the present invention.

When the ceramic composite is processed below 1100 °C, it remains in a weak-field condition with Δₒ<P. Under these conditions, the five unpaired electrons in the Fe³⁺ complex still singly occupy the five 3d orbitals, giving the configuration (t_{2g})³(e_{g})² despite the lifted degeneracy. Thus, even if phonon-activated dynamic J-T distortions invert the energies of the d_{xy} and d_{xz}, d_{yz} orbitals, no photons are emitted because single occupancy blocks electron transitions under the Spin Selection Rule.

Similar considerations apply to [CrO₆]³⁺ complexes, but with a different outcome. Cr³⁺ has a 3d³ electronic configuration in octahedral coordination. When calcined below 1100 °C, its three unpaired electrons continue to singly occupy the three t_{2g} orbitals, giving the configuration (t_{2g})³(e_{g})⁰ after degeneracy is lifted. Thus, the three d electrons remain distributed among d_{xy}, d_{xz}, and d_{yz}. Even if phonon-activated dynamic J-T distortions invert the energies of d_{xy} and d_{xz}, d_{yz}, spin transitions remain forbidden by the Spin Selection Rule, so no photons are emitted.

A spin transition in the 17,000-22,000 cm⁻¹ band is characteristic of octahedrally coordinated low-spin (LS) Cr³⁺. When the ceramic composite is processed above 1100 °C, the crystal-field splitting is about 17,500 cm⁻¹ (Δₒ=2.17 eV), corresponding to blue light at a wavelength of 571 nm. Absorption of this light excites an electron from the t_{2g} orbitals to the e_{g} orbital, producing a (t_{2g})²(e_{g})¹ configuration. In the “Z-in” state, the two t_{2g} electrons occupy the lower-energy d_{xy} orbital and the higher-energy d_{xz} or d_{yz} orbital. When the structure shifts to “Z-out,” these orbitals invert, allowing the electron in d_{xy} to relax into d_{xz} or d_{yz} and emit a photon.

Accordingly, the selected powder mixture must be calcined under a predetermined process to form the intended polymerized silicate structure with distorted octahedral compounds in the strong-field, low-spin state required for the FIR luminescence mechanism.

Calcination is a thermal process that reduces porosity in compacted powder particles and consolidates them into a solid mass. At elevated temperatures, material diffuses into pores at grain boundaries, causing the pores to shrink and increasing intergranular contact. The driving force and kinetics of grain growth depend mainly on particle size and grain-size distribution. In general, higher temperatures accelerate all calcination mechanisms. Because grain growth is often governed by surface diffusion, increasing temperature typically promotes densification more than grain growth, thereby enhancing ion diffusion and dispersion at grain boundaries.

Thus, the calcination heating parameters should be selected to promote silicon oxide polymerization and oxygen sharing with aluminum oxide, thereby driving transformation and recrystallization into a phyllosilicate structure. During this process, doped Fe³⁺ and Cr³⁺ ions from iron oxide and chromium oxide can migrate through the crystal lattice and substitute for Al³⁺ at octahedral sites, forming distorted octahedral compounds that serve as the intended 3-16 μm luminescence centers of the present invention.

It is also important to note that cation dopants (e.g., Li⁺, Na⁺, K⁺, Mg²⁺, and Ca²⁺) can redistribute throughout the crystal during recrystallization and become uniformly distributed within the interstitial layers. This redistribution helps form a network of localized electric fields, creating a strong-field environment that significantly affects the proposed electron transitions between d orbitals within the t_{2g} set.

Phase transformation and crystallization during calcination depend on temperature, pressure, composition, time, crystallography, and the heating and cooling rates. Among these variables, temperature is the primary driver of phase transformation, while the hold time strongly influences the kinetics and promotes formation of equilibrium phases. Although suitable conditions vary with composition and particle size, calcination at 1100 °C or higher for at least 2 hours is generally preferred. In some cases, a lower temperature (e.g., 950 °C) may be used if compensated by a longer calcination time.

As previously described, the IR persistent luminescence mechanism relies on distorted octahedral compounds, specifically [FeO₆]³⁺ or [CrO₆]³⁺ complexes, in a strong crystal field that produces crystal field splitting. This configuration allows the ceramic composite to emit nonthermal IR photons in the 3-16 μm range, providing capabilities not disclosed in the prior art.

Thus, the present invention requires three key components: at least 20 wt. % silicon oxide; at least 8 wt. % aluminum oxide together with at least 2 wt. % iron oxide and/or chromium oxide; and at least 2 wt. % metal oxide. These components define the minimum composition needed to form an effective polymerized silicate structure, preferably a phyllosilicate, that supports the IR persistent luminescence mechanism.

Phyllosilicates are sheet-structured minerals. Their formation begins with selecting suitable precursors, typically SiO₂ (silica) and Al₂O₃(alumina), in proportions that provide the required composition. In some embodiments, instead of pure silicon and aluminum oxides, precursor powders may include aluminosilicate phyllosilicate minerals such as kaolinite (Al₂O₃·2SiO₂·2H₂O), montmorillonite (Na,Ca)(Al,Mg)₂(Si₄O₁₀)(OH)₂, and illite (K)(Al,Mg,Fe)₂(Si,Al)₄O₁₀(OH)₂).

The mixture is then heated, optionally under pressure, to crystallize the layered phyllosilicate structure. The required processing time and conditions depend on the targeted properties of the final composite and should be predetermined accordingly.

The phyllosilicate structure may form either a 1:1 (T-O) or 2:1 (T-O-T) layered arrangement, where T denotes a tetrahedral sheet and O denotes an octahedral sheet. These two structures arise from different thermodynamic conditions governed by silica availability, pH, temperature, and cation activity, which determine whether the sheets link in a T-O or T-O-T pattern. In general, 1:1 (T-O) structures form in low-silica, acidic environments, whereas 2:1 (T-O-T) structures form in silica-rich, neutral-to-alkaline hydrothermal environments.

The nonthermal IR emission mechanism of the present invention relies on phonon-induced dynamic Jahn-Teller distortions in transition-metal octahedra, which alternate between “Z-in” and “Z-out” configurations. This mechanism is more consistent with 2:1-type vibrational behavior associated with higher SiO₂ levels than with the greater rigidity and sharper vibrational modes characteristic of 1:1-type, lower-silica, Al-rich structures.

In view of the foregoing and the potential substitution of Al³⁺ by Cr³⁺ or Fe³⁺, the ideal weight ratio of SiO₂ to the combined amount of Al₂O₃, Cr₂O₃, and Fe₂O₃ is preferably 2, and a ratio of between 1.5 and 2.5 is semi preferred, although ratios from 1 to 3 may be used in practice.

Accordingly, the present invention requires a composition comprising at least 20 wt. % silicon oxide, at least 8 wt. % aluminum oxide together with at least 2 wt. % iron oxide and/or chromium oxide, and at least 2 wt. % metal oxide to form an effective nonthermal IR-emitting ceramic composite. The balance may include additional alumina, silica, silicates, aluminosilicates, hematite, magnetite, chromite, and other metal oxides containing lithium (Li), sodium (Na), potassium (K), magnesium (Mg), calcium (Ca), titanium (Ti), manganese (Mn), cobalt (Co), nickel (Ni), copper (Cu), zinc (Zn), zirconium (Zr), and niobium (Nb).

As noted above, the mixture may include pure oxides, minerals, or both. For example, aluminum oxide and silicon oxide may be supplied as pure oxides or from alumina-, silica-, silicate-, or aluminosilicate-based minerals, provided the required ratio is maintained. Likewise, iron oxide and chromium oxide may be supplied as pure oxides or from minerals such as hematite, magnetite, and chromite, so long as the minimum ratio requirement is satisfied.

In one embodiment, the mixture includes about 52 wt. % SiO₂, 20 wt. % Al₂O₃, 16 wt. % Fe₂O₃, and 4 wt. % Cr₂O₃, corresponding to a ratio of 1.3. The remainder may include oxides of charge-balancing cations, such as K₂O, Na₂O, or CaO, as well as minor oxides.

In some embodiments, natural minerals may be used in place of pure oxides. For example, SiO₂ and Al₂O₃ may be proportionally replaced with quartz and phyllosilicates, while Cr₂O₃ and Fe₂O₃ may be supplied by hematite (Fe₂O₃), magnetite (FeO Fe₂O₃), or chromite (FeO·Cr₂O₃). Suitable phyllosilicates include 2:1 clay minerals (TOT), such as illite, talc, and montmorillonite, and 1:1 clay minerals (TO), such as kaolinite and lizardite.

Minerals may offer cost and environmental advantages in some applications. However, pure oxides generally provide better control, consistency, and application-specific performance. The preferred choice depends on the required balance between precision and practicality.

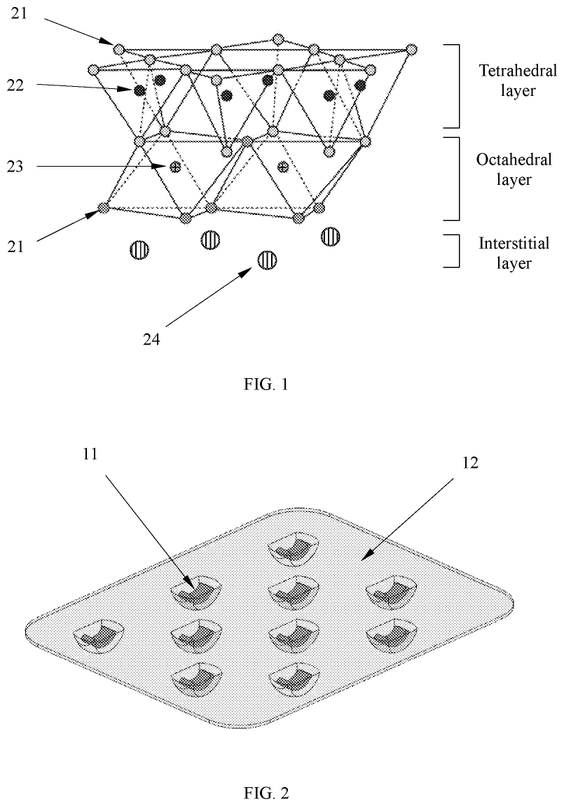

Using the composition and process described above, a ceramic composite of the present invention can be produced that emits nonthermal photons in the 3-16 μm wavelength range. FIG. 1 illustrates the core architecture of the nonthermal IR luminescence mechanism, showing a polymerized silicate structure, preferably a phyllosilicate, with a 1:1 T-O ratio.

Phyllosilicates, or sheet silicates, consist of layered silicon-oxygen tetrahedra and aluminum-oxygen octahedra. In a simple T-O structure, each SiO₄ tetrahedron contains a silicon atom (22) that shares three of its four oxygen atoms (21) with adjacent tetrahedra to form a two-dimensional sheet. This silica sheet is linked through shared oxygen atoms to an alumina octahedral (AlO₆) sheet, in which aluminum ions (23) are coordinated by six oxygen atoms (21). The stacked sheets are stabilized by interlayer cations (24), such as potassium, sodium, and calcium, which balance charge and contribute to the crystal field. During calcination, some octahedral Al³⁺ ions are replaced by Cr³⁺ or Fe³⁺, forming distorted octahedral compounds. Phyllosilicates may also adopt a T-O-T structure.

In addition to phyllosilicates, the polymerized silicates of the present invention may include chain or framework silicates, provided they contain the required distorted octahedral compounds. Polymerized silicates are silicate structures in which SiO₄ tetrahedra are connected by shared oxygen atoms (Si—O—Si bonds). They include a broad range of forms, from dimers and small oligomers to chains, rings, sheets, frameworks, and colloidal or gel networks. The chain structures (inosilicates) include pyroxenes (single chains) and amphiboles (double chains). The 3-D framework structures (tectosilicates) include quartz, cristobalite, feldspars, and zeolites.

In another embodiment, the mixture may include natural mineral powders such as about 50 wt. % kaolinite, 30 wt. % quartz, 12 wt. % magnetite and 3 wt. % chromite, and about 5 wt. % metal oxides. This formulation yields approximately 50-53 wt. % SiO₂, 18-20 wt. % Al₂O₃, 12-15 wt. % Cr₂O₃ and Fe₂O₃, and 5-8 wt. % metal oxides, thereby meeting the minimum requirements of the present invention.

Kaolinite is a clay mineral with the chemical formula Al₂Si₂O₅ (OH)₄. It is a layered silicate composed of one tetrahedral silicate sheet (SiO₄) bonded through oxygen atoms to one octahedral alumina sheet, with adjacent layers held together by hydrogen bonds. Kaolinite typically contains about 48 wt. % SiO₂, 38 wt. % Al₂O₃, and 12 wt. % H₂O, along with minor impurities such as Fe₂O₃, TiO₂, MgO, and CaO.

Quartz is a hard mineral made of silica (silicon dioxide). Its structure consists of a continuous framework of SiO₄ tetrahedra, with each oxygen atom shared by two tetrahedra, giving the overall formula SiO₂. Accordingly, quartz is structurally classified as a framework silicate and compositionally as an oxide mineral.

Hematite is a common iron oxide mineral with the formula Fe₂O₃ and is widely found in rocks and soils. It shares the corundum crystal structure of Al₂O₃ and forms a complete solid solution above 950 °C.

Magnetite is an iron oxide mineral with the chemical formula FeO·Fe₂O₃. It contains both ferrous (Fe²⁺) and ferric (Fe³⁺) iron. As a member of the inverse spinel group, magnetite can form solid solutions with structurally related minerals such as chromite. In both natural and synthetic forms, it commonly occurs as octahedral crystals.

Chromite is an oxide mineral with the formula FeCr₂O₄ (or FeO·Cr₂O₃), composed mainly of iron(II) and chromium(III) oxides. It belongs to the spinel group, forms layered deposits, and can form a complete solid-solution series with related spinel minerals such as magnetite.

This embodiment shows that the composition of the present invention may be made from either pure oxides or minerals that supply the required components. However, composition alone is not sufficient; the material must also be processed to establish the nonthermal IR photon-emission mechanism. Specifically, the process must produce at least one crystal structure containing transition metal ions (Fe³⁺, Cr³⁺) in distorted octahedral sites to enable crystal field splitting and achieve nonthermal photon emission in the 3-16 μm range.

The ceramic composite of the present invention has broad applications, including therapeutic uses in humans and animals.

Referring again to FIG. 2, there is illustrated a preferred embodiment of the present invention in which multiple ceramic composites (11) are embedded in a substrate (12). The substrate may be made of silicone (polydimethylsiloxane), zinc sulfide, sodium chloride, potassium bromide, or similar materials. It is generally a flat sheet with a series of pockets, or curved recesses, sized to hold the ceramic composites. In this embodiment, the pockets are uniform in size for composites of the same dimensions, although other embodiments may use pockets of different sizes or shapes to suit particular applications or composite arrangements. The assembly may also include a flexible attachment mechanism for securing it to the body part being treated.

Forming the ceramic composite (11) with a concave shape, as shown in FIG. 2, may be beneficial. Because ceramic composites emit nonthermal IR in all directions, a concave surface may concentrate the radiation toward a specific area and improve penetration into the body with less attenuation than a flat surface. This focused emission may provide greater therapeutic benefit than a flat ceramic of similar size and mass. Suitable concave forms include hemispherical, bowl-shaped, and partial cylindrical designs. For best results, the concave surface should face the body part being treated. When the device is wrapped closely around the body, the radiation may focus and penetrate greater than one inch into the tissue, thereby enhancing the therapeutic effect.

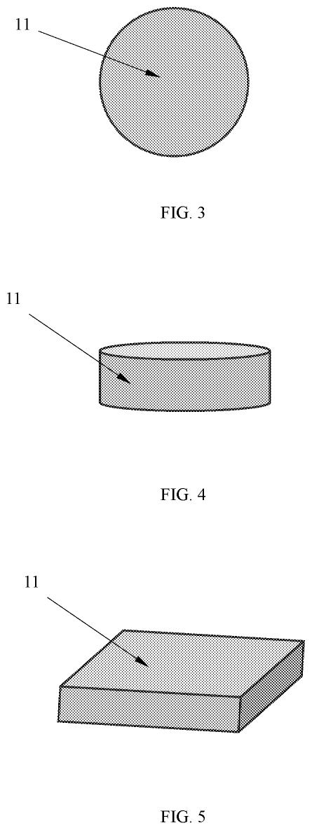

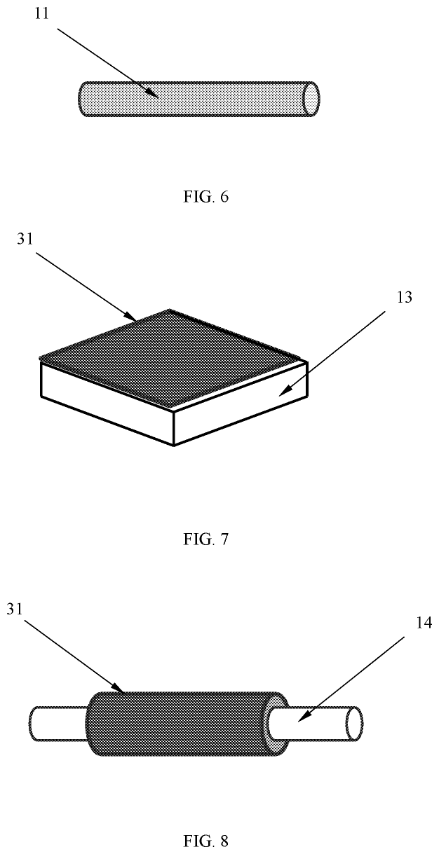

The ceramic composites of the present invention may be formed in various shapes and sizes for different applications. In one embodiment, they are circular, with diameters of 2-50 mm and thicknesses of 1-10 mm. In another embodiment, they are rectangular, with dimensions of 2×3 mm to 40×50 mm and thicknesses of 1-10 mm. Circular and rectangular forms are generally easier to fabricate than other shapes. FIGS. 3-6 illustrate four alternative embodiments in which the ceramic composite (11) is formed as a sphere, a circular plate, a rectangular plate, and a rod or wire, respectively.

The ceramic composite of the present invention may be used directly in a shaped article or ground into submicron particles, preferably larger than 50 nanometers (nm). These particles may be embedded in polymeric or elastomeric materials, such as synthetic resin, polyvinyl chloride, silicone rubber, polyurethane, polystyrene, polyethylene, or similar substances, to form articles such as foam, pads, sheets, mats, beads, or plates in shapes including rectangular, circular, cylindrical, or spherical, as shown in FIGS. 3-6.

Alternatively, the particles may be deposited on a base material such as plastic, rubber, metal, wood, or ceramic, or coated onto fiber or cloth to broaden their applications.

In another embodiment, as shown in FIG. 7, ceramic composite particles (31) are deposited on a base material (13). The base material may be flexible, such as cloth, plastic, or rubber, or rigid, such as wood, ceramic, or metal. The device may also include flexible attachment features, such as straps or adhesives, to secure it to the treated subject.

Additionally, ceramic composite particles (31) may be coated onto wires or fibers (14), as shown in FIG. 8, to expand their applications.

The embodiments described above are passive devices. The ceramic composite absorbs ambient heat, which triggers phonon-activated dynamic J-T distortions and enables photon emission in the 3-16 μm range. This nonthermal IR emission continues as long as the ambient temperature remains above absolute zero. In therapeutic applications, body heat provides an effective source for generating nonthermal 3-16 μm wavelength photons.

These features make the therapeutic device of the present invention simple, effective, user-friendly, and maintenance-free. It requires no external power source and operates solely on body heat.

Nonthermal IR emission in the 3-16 μm wavelength range can be verified using the test methods developed by the present inventor in, for example, aforementioned US Patent Publication Nos. 2024/0003747 and 2025/0314531, which distinguish it from background thermal radiation. This distinction sets the present invention apart from prior art based on thermal radiation.

Exploratory ceramic composite samples were developed to verify the nonthermal IR emission disclosed by the present invention in the 3-16 μm range.

A test sample containing SiO₂ (59.8%), Al₂O₃ (26.9%), Fe₂O₃ (2.3%), NaO (6.0%), K₂O (1.6%), CaO (1.1%), and other impurities was prepared to demonstrate the concept using the minimum iron oxide content (>2 wt. %) needed to achieve effective nonthermal IR emission in the present invention.

Additional samples containing higher wt. % of iron oxide and chromium oxide were prepared to evaluate therapeutic effects, including reduced pain and inflammation, improved circulation, and faster cellular recovery. The emitted 3-16 μm wavelength photons induce intracellular vibrations that support the body's natural healing processes. Promising results were observed in mouse models and clinical trials, particularly for wound healing and for treating neurodegenerative disorders such as Parkinson's disease (PD) and Alzheimer's disease (AD).

According to the present invention, a ceramic composite can emit nonthermal infrared photons in the 3-16 μm wavelength range for broad applications, including therapeutic devices for treating human and animal bodies to improve physiological performance, immune function, overall health, and lifespan. Several embodiments of the inventive composites have been described above, and many modifications and variations may be made in light of these teachings. Such changes do not depart from the spirit or scope of the invention. All modifications apparent to those skilled in the art are intended to fall within the scope of the following claims.

1. A ceramic material configured to emit nonthermal infrared radiation in a wavelength range of 3-16 μm for broad applications, the ceramic material selected from the group consisting of:
at least 20 wt. % silicon oxide (SiO₂), at least 8 wt. % aluminum oxide (Al₂O₃) in combination with at least 2 wt. % of iron oxide (Fe₂O₃), chromium oxide (Cr₂O₃), or a combination thereof, and at least 2 wt. % of metal oxide; and
wherein a mixture comprising the silicon oxide, the aluminum oxide, the iron oxide and/or the chromium oxide, and the metal oxide is calcined according to a predetermined process to form a ceramic composite comprising at least one crystal structure exhibiting crystal field splitting sufficient to permit electron transitions that result in emission of nonthermal infrared photons within the wavelength range of about 3-16 μm.

2. The ceramic material of claim 1, wherein:
the metal oxide comprises one or more of the following elements: lithium (Li), sodium (Na), potassium (K), magnesium (Mg), calcium (Ca), titanium (Ti), manganese (Mn), cobalt (Co), nickel (Ni), copper (Cu), zinc (Zn), zirconium (Zr), and niobium (Nb).

3. The ceramic material of claim 1, wherein:
the mixture contains at least one of a pure oxide or a minerals.

4. The ceramic material of claim 3, wherein:
the mineral comprises at least one of a silica, a silicates, an aluminosilicates, an alumina, a hematite, a magnetite, or a chromite.

5. The ceramic material of claim 1, wherein:
the process includes calcining at a temperature of 1100 °C or higher.

6. The ceramic material of claim 1, wherein:
the ceramic composite is formed into a shaped article comprising any suitable shape, wherein the shape is selected from a group consisting of rectangular, circular, cylindrical, or spherical shapes.

7. The ceramic material of claim 1, wherein:
the ceramic composite is reduced to particles and applied to a material to form a shaped article.

8. The ceramic material of claim 7, wherein:
the ceramic particles are incorporated into a polymeric or elastomeric material to form a shaped article, wherein the shaped article Is selected from a group consisting of foam, pads, sheets, mats, beads, or plates.

9. The ceramic material of claim 7, wherein:
the ceramic particles are deposited on a substrate.

10. The ceramic material of claim 7, wherein:
the ceramic particles are coated onto a fibrous or textile material.
